(19) United States
(12) Patent Application Publication
(10) Pub. No.: US 2006/0282109 A1
(43) Pub. Date: Dec. 14, 2006

(54) Blood collection system

(76) Inventors: Paul Jansen, Mannheim (DE); Juergen Rasch-Mengens, Schwetzingen (DE)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 10/560,226

(22) PCT Filed: Apr. 28, 2004
(86) PCT No.: PCT/EP04/04467

§ 371 Date: Dec. 12, 2005

(30) Foreign Application Priority Data
Jun. 13, 2003 (DE) 103 26 692.5

(51) Int. Cl. A61B 17/32

(52) U.S. Cl. 606/181

(57) ABSTRACT

In order to be able to reuse the lancets (5) that are stored in a lancet storage container (6) and used in a blood withdrawal system for taking a blood sample for analytical and diagnostic purposes, and in order to reduce the time period elapsing until the puncturing motion is performed, it is proposed to provide a lancet tip protective element (13) in a parking position of the lancets (5) by means of which the lancet tip is protected mechanically and hygienically.

The invention relates to a blood withdrawal system for taking blood samples for analytical or diagnostic purposes.

For qualitative and quantitative analysis of components of a liquid sample, in particular of a body fluid from humans or animals, test methods involving test elements are being used extensively. The test elements contain reagents. The test element is contacted with the sample to perform a reaction. The reaction between sample and reagent leads to a change in the test element that is characteristic for the analysis and is analyzed with a suitable analytical device. Usually, the analytical device is suitable for the analysis of a specific type of test elements made by a specific manufacturer. The test elements and the analytical device are mutually adapted components and, in combination, are called analytical system.

Numerous different types of test elements are known which differ from each other by their measuring principle and the reagents used as well as by their set-up.

With regard to the measuring principle, calorimetric analytical systems are particularly common. In these systems, reaction of the sample with the reagents contained in the test element leads to a color change that can be measured visually or by means of a photometric measuring facility. Moreover, electrochemical analytical systems have gained great significance, in which the reaction of the sample with the reagents of the test element leads to an electrically detectable change (of an electrical voltage or an electrical current) that is measured with appropriate measuring electronics.

With regard to the set-up of the test elements, strip-shaped test elements (so called test strips) consisting essentially of an elongated carrier layer made of a plastic material and test fields applied thereto are particularly common. The test fields usually consist of test layers containing one or several reagents. Such test strips are used extensively, in particular for blood and urine analysis.

In a second type of test element, a test field is surrounded by a frame similar to a photographic diapositive. The test field of this type of test element usually consists of one or several test layers that are held by the frame and contain suitable reagents for calorimetric tests. After the sample is applied to the test field and the reaction has proceeded, the generation of color can be observed or measured by photometry.

Lancets to be punctured into the respective body part are used to obtain a small quantity of blood from a part of the body (usually the finger or ear lobe) for analytical-diagnostic purposes. In as far as the lancets are punctured into the skin by hand in order to generate a wound, specially trained personnel is required for this purpose. However, puncturing is associated with considerable pain.

Blood withdrawal systems consisting of a puncturing device and corresponding lancets that are specifically adapted to the corresponding device have been in use for a long time. A housing of the puncturing device contains a lancet drive by means of which a lancet is punctured mechanically through the skin. Although this process also is not completely free of pain, there are various developments aiming to render the taking of a blood sample as painless as possible.

This is desired in particular when regular monitoring of certain analytic blood values is required. This applies in particular to diabetics who should monitor their blood sugar levels frequently in order to keep these levels (ideally at all times) within certain nominal limits by suitably adapting their insulin injections to the strongly varying needs.

However, in its practical use, a blood withdrawal system is not only expected to meet the requirement of minimal pain sensation, but also has to be easy to operate, have a compact, slim design, and be easy and cheap to manufacture. These practical requirements have lead and are leading to the development of blood analysis devices, which aim to satisfy these, to some extent contradictory, requirements to the extent possible.

Especially in the area of so-called “home monitoring”, i.e. where medical laymen perform simple blood analyses, and, in particular, in the periodical taking of blood samples several times daily by diabetics to check their blood glucose concentration, lancets and matching devices, so called “puncturing aids”, are available that facilitate the taking of blood samples with as little pain and as reproducibly as possible.

Whereas clinics and physicians in private practice often take several milliliters of blood samples from a person to be tested by venous puncture for subsequent analysis to have a multitude of laboratory tests conducted on these samples, individual analyses targeted at one certain parameter nowadays require no more than a few μl of blood. The collection of small sample volumes in the range of few μl or less for the determination of analytical parameters is common especially in blood glucose monitoring, but is also applied in the determination of coagulation parameters, triglycerides, HbA 1c or lactate.

Such small quantities of blood do not necessitate venous puncture, but can be taken with a sterile, sharp lancet that is punctured through the skin, e.g. into the tip of the finger or the ear lobe of the person to be tested. This method is particularly well-suited in cases, in which the blood sample is analyzed immediately after taking the sample.

The lancets used to take body fluids from a part of the body by generating a small puncture wound usually possess a metal lancet needle, the tip of which may be beveled. These lancets must be stored under sterile conditions until use, and preferably should be disposed after use such that they cannot cause injury. Therefore, blood withdrawal systems were proposed, in which the lancets are contained in a lancet storage container, in which a plurality of lancets is kept ready for their removal from the lancet container at a removal position.

One possible embodiment of a lancet storage container of this type is a drum cartridge, from which the lancets can be removed individually, whereby the lancets are arranged in individually closed chambers inside the drum cartridge. The used lancets are then disposed of either outside the device and/or analytical device or can be returned to the lancet storage container for safe disposal.

The document, DE 198 40 856 A1, describes a lancet dispenser for a blood withdrawal system and a corresponding method for removing a lancet from a lancet cartridge. The lancet dispenser comprises a housing with a lancet storage container being arranged therein, a lancet guide, and a lancet drive.

In order to meet the requirements mentioned above, the current development of blood analysis devices also aims to provide highly integrated devices, in which all functions preferably are combined. Blood analysis devices of this type, glucose meters, for example, comprise in a device that is as compact as possible both a blood withdrawal system with lancets, a cartridge for the lancets, an analytical facility and/or analytical system, and the requisite test elements, which preferably are also provided in a cartridge. In this context, it is preferable for all materials that are provided in a cartridge, i.e. the lancets and the test elements, to be disposed of inside the device after their use, for example, to be returned to the cartridge after use and stored therein until replacement of the entire cartridge.

For example, another known concept of blood analysis devices is based on the use of integrated “disposables”, i.e. an integrated combination of one lancet and one test element each. This means that one lancet each, integrated into the test element, is provided or present for each test element.

A number of questions arise with regard to such integrated or highly integrated measuring devices of this type, in particular devices for mobile use such as in home monitoring:
According to the prior art, a fresh lancet is used each time a blood sample is taken. As a result the lancet cartridge requires more of the volume of the blood analysis device than the test element cartridge. In order to reduce the size of the blood analysis device, it is desirable to reduce the volume of the lancet storage container.
In some cost reimbursement systems, only the costs of disposables required to perform the analysis, for example the test elements or test carriers used, are reimbursed, but not accessory parts, such as lancets. This leads to the wish to use lancets multiply for taking blood samples, i.e. use lancets for several puncture motions.
With regard to the multiple use of lancets, there is not only a need to protect the lancets mechanically and hygienically prior to their first use, but also to protect the lancet mechanically and hygienically until its reuse. Any residual blood or skin remaining on the lancet from its first use should be removed in the process.
And lastly, a lancet that is disposed after its final use in the lancet storage container or by other means or outside of the blood analysis device and/or blood collection system should be prevented from being contaminated and thus posing an ensuing risk of contamination to the unused lancets kept in store in the lancet storage container.
Another problem with highly integrated blood analysis devices is that the time period between the start of a measuring process and the actual puncturing of the skin can be relatively long. From the time, at which a measuring process is started, it may take up to 10 seconds before the puncturing motion into the skin is performed by the lancet. The site of the body to be punctured, for example the finger, must be pressed against the exit opening of the blood analysis device for this period of time, and it must be waited for the puncturing motion.
Many users consider this period of time excessive. However, it cannot be reduced significantly with the blood collection systems that are common according to the prior art, since this period of time is needed by the lancet drive to remove a lancet from the lancet storage container and transport it to the puncturing position on the exit opening for the lancet tip.
The present invention aims to solve the problems associated with the multiple use of lancets encountered with previously known blood withdrawal systems.
This object is met according to the invention by a blood withdrawal system and/or a method for taking a blood sample having the features of the appended independent patent claims. Preferred embodiments and developments of the invention are evident from the dependent patent claims and the following description with corresponding drawings.
A blood withdrawal system according to the invention for taking blood samples for analytical or diagnostic purposes thus comprises a housing with an exit opening for the lancet tip of the lancet needle of a lancet that can be moved in the housing along a predetermined puncturing path, a lancet guide by means of which the lancet can be guided along the predetermined puncturing path, a lancet drive by means of which the lancet can be driven and moved along the predetermined puncturing path in the puncturing direction until its tip, being in a puncturing position, exits from the exit opening, and a lancet storage container that is arranged in the housing and in which a plurality of lancets is kept in store at a removal position for removal from the lancet storage container, and is characterized in that it comprises a lancet tip protective element, into which the lancet tip can be inserted before or after a puncturing motion, whereby the protective element mechanically and hygienically protects a lancet tip that is inserted therein, and in that the lancet tip protective element is arranged on the lancet tip in a parking position of the lancets, whereby the parking position does not coincide with the removal position and the puncturing position.
A method according to the invention for taking blood samples with a blood withdrawal system for analytical or diagnostic purposes, whereby the blood withdrawal system comprises a housing with an exit opening for the lancet tip of the lancet needle of a lancet that can be moved in the housing along a predetermined puncturing path, comprises a lancet guide by means of which the lancet can be guided along the predetermined puncturing path, comprises a lancet drive by means of which the lancet can be driven and moved along the predetermined puncturing path in the puncturing direction until its tip, being in a puncturing position, exits from the exit opening, and comprises a lancet storage container that is arranged in the housing and in which a plurality of lancets is kept in store at a removal position for removal from the lancet storage container, is characterized in that the lancet tip is inserted into a lancet tip protective element before or after a puncturing motion, whereby the protective element mechanically and hygienically protects a lancet tip that is inserted therein, and in that the lancet tip protective element is arranged on the lancet tip in a parking position of the lancets, whereby the parking position does not coincide with the removal position and the puncturing position.
The particularity of the invention is essentially to be seen in that, aside from a storage position (within the lancet storage container and/or in its removal position) and an application position (in the puncturing position on the exit opening), an additional standby position (the parking position) is provided for the lancets, in which they are inserted into a lancet tip protective element and protected by this element.
The first use of a lancet for taking a blood sample can proceed either in common fashion by removing the lancet from the lancet storage container and guiding it directly to the exit opening. Alternatively, it is also feasible to provide the lancet in the parking position ready for use prior to its first use. However, the repeated use of the lancet always starts from the parking position to which it is transported after its first use and the subsequent uses until final disposal. After the final use of a lancet for taking blood samples, the lancet can either be disposed of directly, for example within the lancet storage container or outside of the device, or it can be returned again into the parking position prior to disposal in order to clean it there and prevent any contamination of other elements, for example the lancet storage container.
The invention allows lancets, including those in blood analysis devices, to be used multiply and to protect them mechanically and hygienically prior to their application, i.e. puncturing of the skin, whereby simultaneously the period of time between starting a process of taking a blood sample and performing the puncturing process can be kept short. In the parking position, the lancet can be cleaned to a great extent, for example by sterilization, and at the same time it is protected from environmental influences, e.g. shocks.
While the lancet is transiently arranged in the parking position, it can be kept therein ready for use. If the arrangement and/or the path of motion of the lancet is optimized such that its feeding forward from the parking position to the puncture position on the exit opening can proceed more rapidly in the blood analysis device than from the removal position in the lancet storage container, the period of time elapsing until the puncturing motion is performed can be reduced as desired.
Accordingly, a further advantageous feature of the invention proposes to arrange the lancet tip protective element and the parking position in the immediate vicinity of the exit opening.
A mechanically simple and thus preferred embodiment consists of the lancet tip protective element being arranged in a stationary position, i.e. in a fixed place in the housing of the blood collection system and/or blood analysis device. However, other embodiments can provide for the lancet tip protective element to be driven by means of a lancet tip drive.

According to a further preferred feature it is proposed that the lancet tip protective element comprises an elastic material, into which the lancet tip can be inserted. This can, for example, be a plastic body consisting of an elastic material in the area of the lancet tip. At least the tip of the lancet needle is completely surrounded by the elastic material on all sides, i.e. it is embedded therein and thus sealed off from its surroundings. The elastic material is characterized, for example, by being soft, pliable, and capable of being penetrated by the tip of the lancet is needle without damaging the tip of the lancet. A further important feature of the elastic material is that it re-closes upon retraction of the lancet needle from the lancet tip protective element, if applicable, such that the lancet needle can be re-inserted after a puncturing process or another lancet needle can be inserted into the elastic material.

The elastic material provides mechanical protection to the lancet tip and thus prevents damage to the lancet tip. By surrounding the lancet tip, it also contributes to the sterility of the lancet tip prior to its use, in particular if it is sealed tight with regard to the penetration or escape of pathogens as it may be depending on whether the lancet needle was used previously or not.

Suitable elastic materials are described in the documents, WO 01/66010 A1 and US 2001/0041904 A1. Reference shall thus be made to these documents. Suitable elastic materials include, for example, silicone, rubber and/or elastomers (e.g. polybutadiene or isoprenes such as polyisoprene=India rubber, possibly vulcanized to increase its hardness), elastomericcopolymers (e.g. styrene-butadiene copolymers) or thermoplastic elastomers (e.g. polyurethanes). They are soft, pliable, can be penetrated by the lancet needle without damaging the lancet tip, and closely surround the used lancet tip. Moreover, thermoplastic elastomers can be processed by injection molding processes and are thus cheap to manufacture.

Within the elastic material, for example a soft plastic material, preferably with germicidal/microbicidal properties, the lancet tip can reside protected and is stored in a sterile fashion prior to its first use or between applications. The soft plastic material should be soft enough for the lancet tip or the lancet not to be damaged and excessively blunted upon being inserted into the material. At the same time, the material should be viscous enough to still surround, i.e. seal, the lancet even after multiple applications without becoming brittle.

A further advantageous design consists of the lancet tip protective element comprising a sterilizing, microbicidal, inactivating, disinfecting, bactericidal or fungicidal material for cleaning or protecting the lancet tip. In this context, it is preferred for the elastic material to be designed as described. The lancet is then protected and cleaned in its parking position, and can thus be used multiply.

Suitable sterilizing materials are disclosed in the document, US 2001/0041904 A1, reference to which is thus being made. Suitable materials include, for example, oxidants, phenols, epoxides, peroxides, polymers of formaldehyde and other mono-, di-, and polyaldehydes (e.g. glutaraldehyde), hydrogen peroxides-containing systems, iodine-containing complexes, and metal salts of silver or copper. With regard to the invention, the SAM polymers of Degussa have proven to be particularly advantageous, for example Limago T100 or Amina T100, which can be admixed to a plastic material.

A blood withdrawal system according to the invention is preferably designed such that a lancet can be used multiply to take a blood sample and can be moved into the lancet tip protective element between the puncturing motions. Because of the multiple use of lancets, a blood collection system and/or a corresponding blood analysis device according to the invention can be designed such that the number of lancets provided is smaller than the number of corresponding test elements rather than, as according to the prior art, being equal to the number of the corresponding test elements that are provided. This permits both space and cost savings to be achieved.

It is advantageous to provide operating elements the user can use to set whether a new lancet from the lancet storage container or a lancet from the parking position in the lancet tip protective element that was used previously for taking a blood sample is used for the subsequent blood collection process. By this means, the user can decide about the point in time at which he wishes to use a new lancet when the sharpness of the lancet tip decreases and the ensuing pain upon puncturing increases. This can, for example, also be implemented by the user pre-setting the number of blood samples to be taken with one lancet and the lancet being disposed of after this pre-set number of applications is reached.

The invention proves to be advantageous especially as a highly integrated analytical system, whereby the system includes test elements in addition to a lancet cartridge. Providing both lancet and test elements in the system causes the lancet to have to undergo an extensive sequence of motions to effect its exiting from the housing—as described above—since the lancet cartridge cannot be positioned directly above the opening of the housing. Accordingly, a complex sequence of motions in X and Y direction is required to perform a puncturing process, whereby this sequence of motions can be simplified by providing the parking position described above. Therefore, according to an advantageous feature, it is proposed that the blood withdrawal system comprises a test element cartridge that is integrated into the blood withdrawal system, preferably by being arranged in the housing. It is preferable for the test element cartridge to be replaceable.

The invention is illustrated in the following based on exemplary embodiments shown in the figures. The particularities illustrated therein can be used separately or in combination in order to create preferred developments of the invention. In the figures:

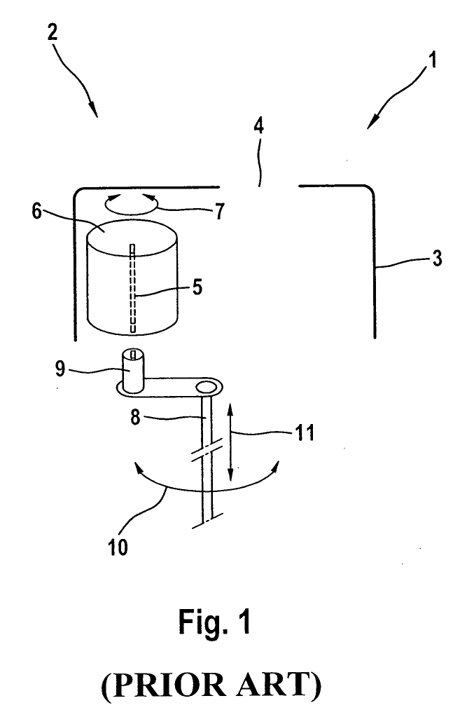

FIG. 1 shows the set-up of a glucose monitor according to the prior art;

FIG. 2 shows step 1 of a sample taking process according to FIG. 1;

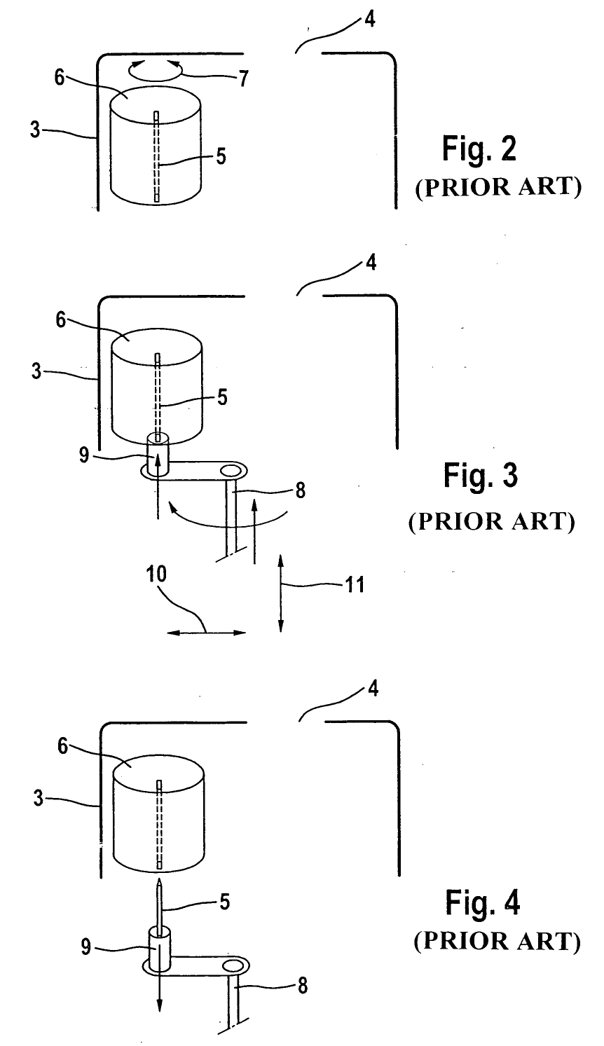

FIG. 3 shows step 2 of a sample taking process according to FIG. 1;

FIG. 4 shows step 3 of a sample taking process according to FIG. 1;

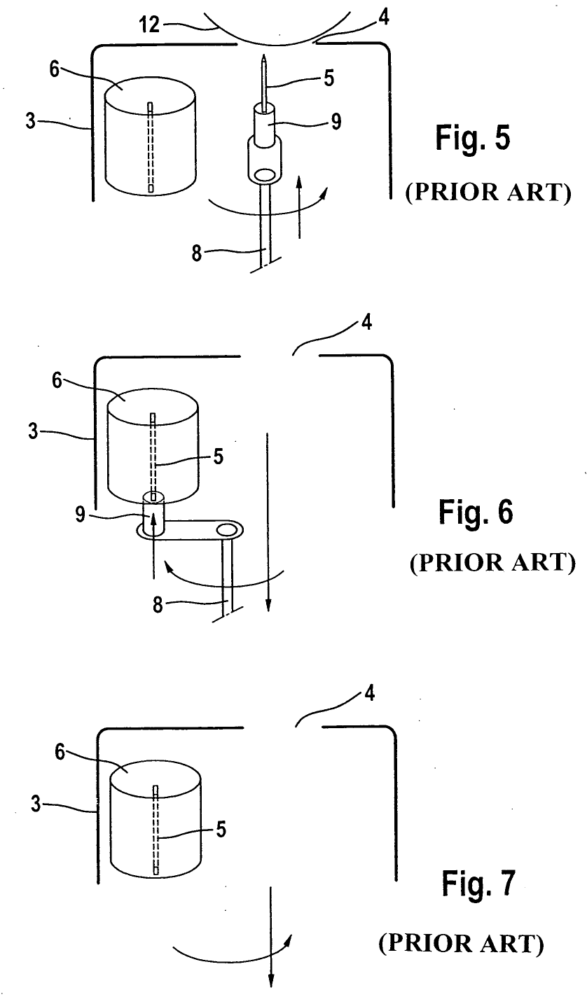

FIG. 5 shows step 4 of a sample taking process according to FIG. 1;

FIG. 6 shows step 5 of a sample taking process according to FIG. 1;

FIG. 7 shows step 6 of a sample taking process according to FIG. 1;

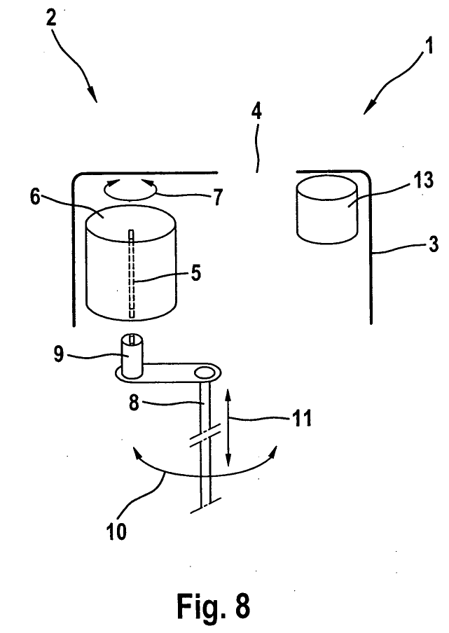

FIG. 8 shows the set-up of a glucose monitor according to the invention;

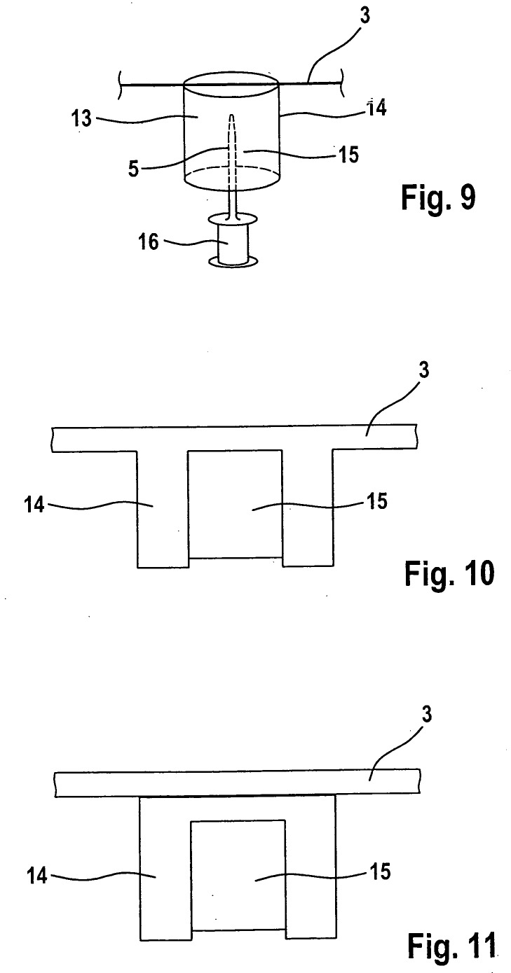

FIG. 9 shows a detail related to FIG. 8;

FIG. 10 shows another detail related to FIG. 8;

FIG. 11 shows a modification related to FIG. 10;

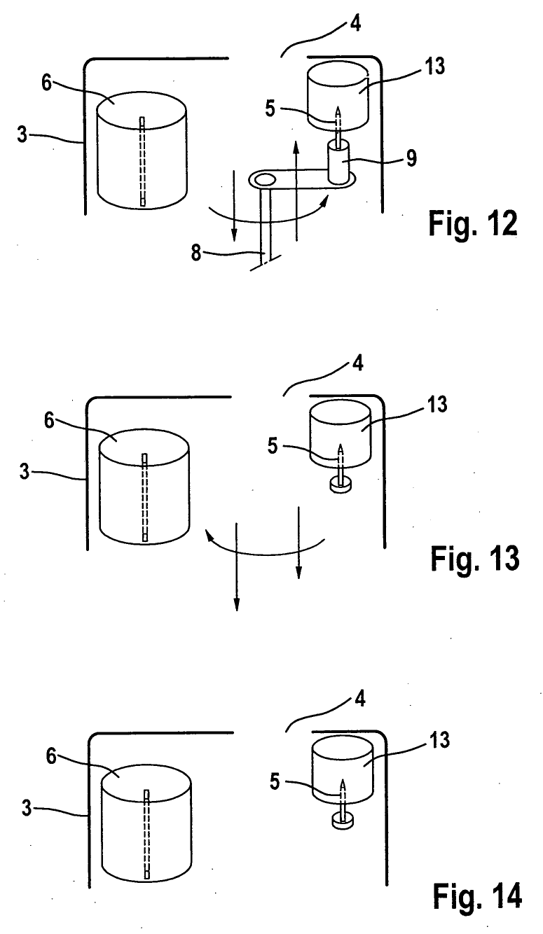

FIG. 12 shows step 1 of a sample taking process according to FIG. 8;

FIG. 13 shows step 2 of a sample taking process according to FIG. 8;

FIG. 14 shows step 3 of a sample taking process according to FIG. 8;

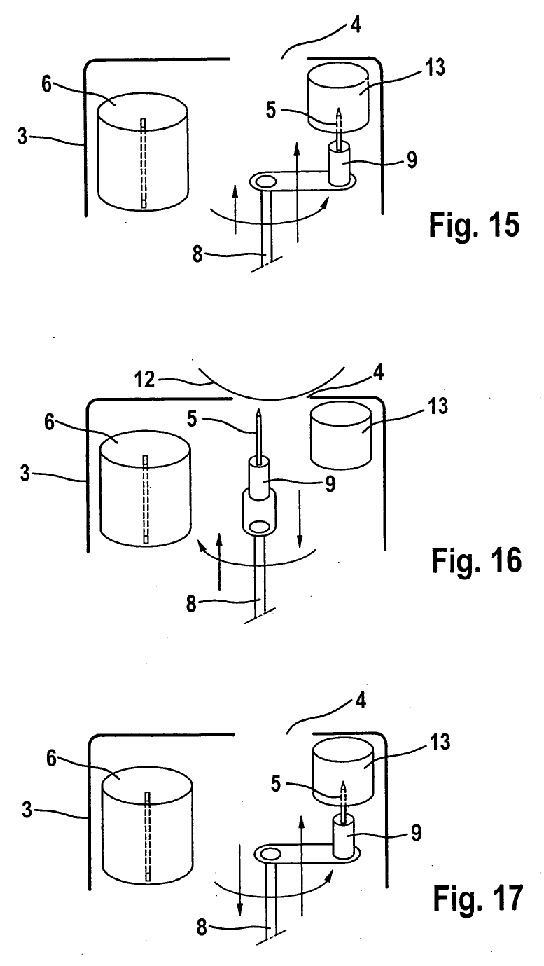

FIG. 15 shows step 4 of a sample taking process according to FIG. 8;

FIG. 16 shows step 5 of a sample taking process according to FIG. 8;

FIG. 17 shows a modified step 1 of a sample taking process according to FIG. 8;

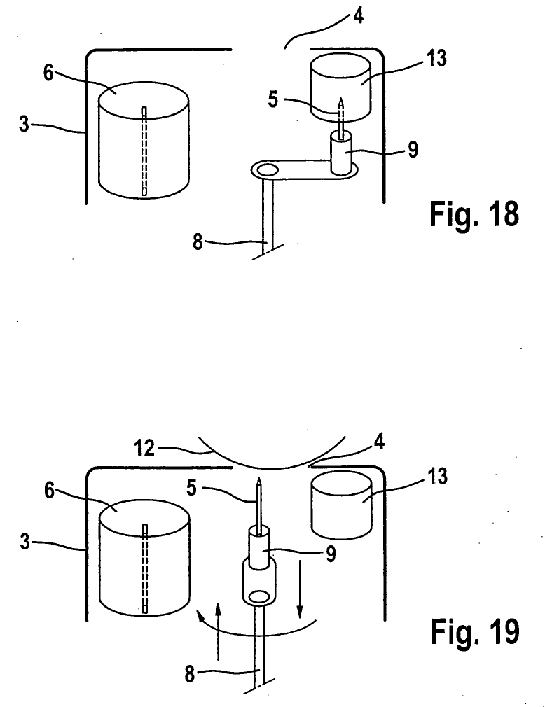

FIG. 18 shows a modified step 2 of a sample taking process according to FIG. 8;

FIG. 19 shows a modified step 5 of a sample taking process according to FIG. 8;

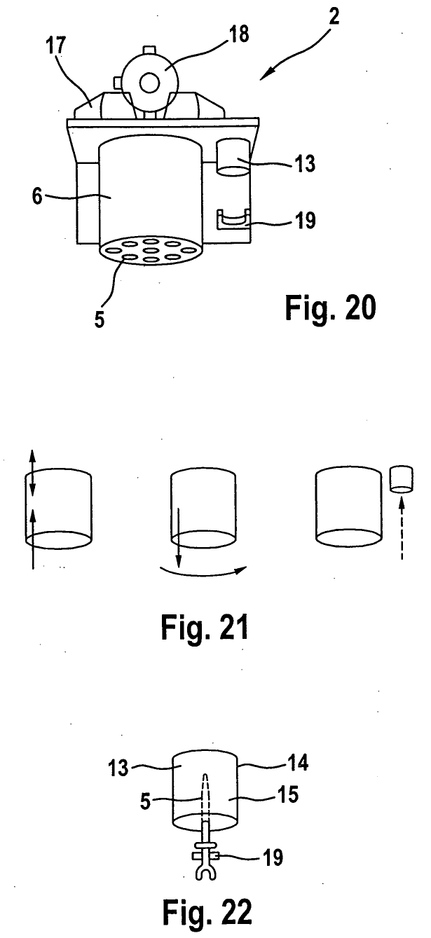

FIG. 20 shows a modification related to FIG. 8,

FIG. 21 shows a detail related to FIG. 20,

FIG. 22 shows another detail related to FIG. 20; and

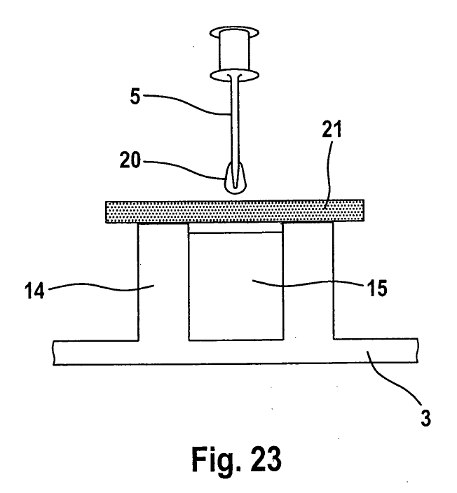

FIG. 23 shows another modification related to FIG. 10.

FIG. 1 illustrates schematically the blood withdrawal system 1 of a blood analysis device 2 according to the prior art in the form of an integrated glucose monitor, in which the disposables required for blood analysis are kept in a cartridge and in which the processes required for blood analysis are integrated. For reasons of clarity, the test elements used to analyze the blood sample are not shown.

The blood withdrawal system 1 comprises a housing 3 with an exit opening 4, at which a blood sample can be taken by means of a lancet 5. The lancets 5 reside in a lancet storage container 6 that is provided in the form of a drum cartridge capable of rotation in the exemplary embodiment shown. The multiple lancets 5 are kept in the lancet storage container 6 in the form of a cartridge and can be provided therein either loose or clamped by holding elements. The lancet storage container 6 can be rotated about a rotation axis 7 in order to move to a removal position a lancet 5 that is contained in the lancet storage container 6 and is to be removed.

Through the use of a lancet drive 8, provided, for example, in the form of a lift-pivot unit with an adapted grabber 9, a lancet 5 can be removed from the lancet storage container 6 and transported to the sample collection site, i.e. to the puncturing position on the exit opening 4, and transported back to the lancet storage container 6 for disposal after the blood taking process. The lancet drive 8 can be rotated about a rotation axis 10 and moved along a linear axis 11 that is oriented along the puncturing direction.

The procedure of taking a sample with a blood withdrawal system 1 according to the prior art according to FIG. 1 is illustrated in the following figures. Firstly, according to FIG. 2, the lancet storage container 6 rotates such that a lancet 5 to be removed, preferably the one from the next available position, is moved to the 5 removal position, in which it can be grabbed by the grabber 9 of the lancet drive 8. For this purpose, according to FIG. 3, the lancet drive 8 first moves upwards from a starting position that is not shown to a rotation position, then it rotates the grabber 9 about the rotation axis 10 into the reception position, i.e. underneath the position of the lancet 5 to be removed from the lancet storage container 6, and then the lancet 5 is attached to the grabber 9 by another driving motion in upward direction. The downward motion shown in FIG. 4 then pulls the lancet 5 from the lancet storage container 6 with lancet 5 thus arriving in a position, in which it can be rotated about the rotation axis 10.

According to FIG. 5, the lancet 5 is then positioned underneath the exit opening 4 for taking a sample by means of a rotation motion, and the lancet 5 is driven upwards to and/or into the exit opening 4 by means of a driving motion, where it then resides in the puncturing position. A part of the body, e.g. a finger tip 12, is then placed onto the exit opening 4. By means of a short puncturing motion that is not shown, the lancet tip on the lancet needle of the lancet 5 can puncture the finger tip 12 or the surface of the skin of a different part of a body placed onto the exit opening 4, and obtain a small blood sample.

After the blood sample was taken and analyzed with an analytical facility and/or analytical system that is not shown, for example analyzed by means of test strips, either inside or outside of the housing 3, the lancet 5 is transported back into the lancet storage container 6 according to FIG. 6. For this purpose, the lancet drive 8 first performs a downward motion to the rotation position. Then the lancet drive 8 rotates the grabber 9 with the lancet 5 to be disposed to the lancet storage container 6, namely to the now empty position in the cartridge, and, by means of a subsequent driving motion in upward direction, the used lancet 5 is returned to the lancet storage container 6. Subsequently, the lancet drive 8 again drives a small distance downwards, pivots back according to FIG. 7, and then drives down into the starting position that is not shown, from which the next blood taking process proceeds. For another blood taking process, the steps identified above are repeated, whereby each time a new lancet 5 is removed from the lancet storage container 6 and all motions and paths driven are repeated.

The procedure according to the prior art described in FIGS. 2 to 7 has several disadvantages. For one, the paths driven and thus the driving times of the lancet drive 8 are long such that the user must wait for a long time from the initiation of the blood taking process from the starting position of the lancet drive 8 for a lancet 5 to be provided from the lancet storage container 6 and the puncturing motion on the exit opening 4 to be performed. Moreover, the lancet storage container 6 takes up relatively much space, since a new lancet 5 is provided for each blood taking process. In the case of multiple use of a lancet 5 from the storage container 6, there still is the problem of long paths driven and there are problems related to the sterilization of a used lancet 5 prior to its next use and the prevention of contamination of the lancet storage container 6 containing unused, sterile lancets 5 by a used lancet being introduced therein.

These disadvantages are resolved through a blood withdrawal system 1 according to the invention according to FIG. 8. The set-up corresponds to that of the blood withdrawal system according to the prior art according to FIG. 1 with the difference being that it comprises a lancet tip protective element 13 into which the lancet tip can be inserted before and after a puncturing motion, whereby the lancet tip protective element 13 mechanically and hygienically protects a lancet tip that is inserted therein. In a parking position of the lancets 5, the protective element 13 is arranged on the lancet tip, i.e. the lancet tip is inserted into the protective element 13 in the parking position. The parking position coincides neither with the removal position, in which the lancet 5 is removed from the lancet storage container 6, nor with the puncturing position on the exit opening 4, in which the blood taking process is performed.

The parking position for the lancet tip, i.e. the position of the protective element 13, preferably resides in the immediate vicinity of the exit opening 4, preferably inside the housing 3. Another advantageous embodiment can consist of the lancet tip protective element 13 being arranged inside the lancet storage container 6, i.e. being integrated into the cartridge.

The elastic material 15 is subject to wear and tear upon use, for example the plastic material gets perforated or it gets contaminated (residual blood, skin scales, environmental influences). For this reason, it is advantageous for the lancet tip protective element 13 or the elastic material 15 not to be a permanent, i.e. fixed or lasting, component of the device, but rather be replaceable. One possibility is to design the protective element 13 and/or the elastic material 15 in the form of a disposable, i.e. as consumables, to be replaced by the user at times to be determined, e.g. as a separate article or as an accessory in each package of lancets 5. Another possibility is the integration of the lancet tip protective element 13 into the lancet storage container 6. It is then co-replaced with each change of cartridge and does not mean any added concern to the user due to additional service actions.

FIGS. 9 to 11 illustrate embodiments of protective elements 13 arranged to be stationary, i.e. in a fixed place, in the housing 3. FIG. 9 shows a protective element 13 arranged on the inside of the housing 3. It comprises a solid form body 14 filled with an elastic material into which the lancet tip can be inserted. It is filled with a soft plastic material to which a sterilizing, germicidal agent has been admixed. The elastic material 15 protects the tip of the lancet 5 inserted therein from environmental influences, e.g. contaminations, shocks, and related damage, cleans it by wiping off the contamination when the lancet 5 is immersed into the elastic material 15 and disinfects it by means of the admixed microbicide.

In the parking position of the lancet 5 in the lancet tip protective element 13 shown in FIG. 9, the lancet 5 can be held protected against falling out by the lancet drive 8 and/or at a grabber section 16 by a holding facility that is not shown. A holding facility of this type can work actively, i.e. comprise an active grabbing element, or be provided to be passive, for example in the form of a clamping facility.

FIGS. 10 and 11 illustrate details of FIG. 9. In FIG. 10, the lancet protective element 13 is integrated in the inside of the housing 3; in FIG. 11, it is placed on the inside of the housing 3. Another modification is shown in FIG. 23.

FIGS. 12 to 19 illustrate the procedure of taking a sample with a blood withdrawal system 1 according to FIG. 8. The preceding steps, in which the lancet 5 is removed from the lancet storage container 6 and blood is taken by a puncturing motion into the finger tip, are not shown. These steps proceed analogous to FIGS. 2 to 5. Alternatively, it is also possible to use a new, unused lancet 5, removed from a lancet storage container 6, not directly for taking a blood sample, but first transport it into the parking position in the lancet tip protective element 13 and remove the lancet from the parking position at the actual time when blood is taken. This allows the time period until the puncturing motion is performed with a new, unused lancet to be shortened also.

FIGS. 12 to 19 illustrate the further procedure after a process for taking a blood sample was performed with a lancet 5. In the embodiments according to FIGS. 12 to 16, the lancet 5 remains situated in the parking position in the protective element 13 and the lancet drive 8 drives into a separate starting position. According to FIGS. 17 to 19 the lancet drive 8 stays with lancet 5 in the parking position.

In FIG. 12, the lancet drive with grabber 9 and grabbed lancet 5 is initially driven downward after the puncturing motion. By means of a rotation motion that occurs in a rotation plane that is situated as closely as possible to the rotation plane in which the pivoting motion for pick-up of a lancet 5 from the lancet storage container 13 occurs, the grabber 9 is rotated to be underneath the protective element 13. By means of an upward motion, the lancet 5 is moved into the parking position, whereby it is pressed into the soft plastic material in the protective element 13. Therein, it can be held by means of an active or a passive holding facility that is not shown, if the holding force of the elastic material 15 were to be insufficient.

It is preferable for the lancet 5 to be capable of being driven into its parking position by the lancet drive 8, in which position its tip resides inside the protective element 13. If a greater constructive effort can be tolerated, a separate drive can be provided for this purpose, though this drive does not perform the function of picking-up the lancets 5 from the lancet storage container 6.

The constructive effort and paths driven by the lancet 5 are minimized by arranging the lancet tip protective element 13 such that the lancet tip can be inserted into the lancet tip protective element by a motion that proceeds parallel to the puncturing motion.

In FIG. 13, in which the lancet 5 stays in the protective element 13 without the lancet drive 8, the lancet drive 8 without lancet 5 initially moves a small distance back down, then pivots back into a position in which it is pivoted away and then drives further down to the starting position that is situated beyond FIG. 13.

The resulting final position, in which the lancet drive 8 resides in a starting position beyond the drawing and the lancet 5 resides in the parking position in the lancet tip protective element 13, is shown in FIG. 14. The lancet 5 can be picked up from the parking position by the lancet drive 8 in order to perform another blood collection process. Parking the lancet 5 outside the lancet storage container 6 prevents any contamination of the lancet storage container 6 or of unused lancets contained therein by sample material adhering to the used lancet 5. Moreover, the transport of the lancet from the parking position to the exit opening for another puncturing process can proceed more rapidly, since the lancet storage container 6 does not need to be repositioned for the removal of a new lancet out of it.

FIGS. 15 and 16 illustrates how a lancet 5 that has been used previously one or more times (or an unused lancet 5, as the case may be) is picked-up from the parking position in the protective element 13 for taking a blood sample, and disposed thereafter. According to FIG. 15, the lancet drive 8 initially drives from the starting position upwards, then pivots the grabber 9 to be underneath the lancet 5 in its parking position, and drives a small distance upwards in order to grab the lancet 5. Subsequently, according to FIG. 16, the lancet drive 8 drives a short distance downwards, whereby the lancet 5 is pulled from the protective element 13, pivots towards the left and performs an upward motion to move the lancet 5 into the puncturing position on the exit opening 4 in order to puncture it into the finger tip 12.

When the lancet 5 is no longer needed, for example because the number of puncturing processes to be performed with a lancet 5 as set by the user has been performed, the lancet 5 is transported to the lancet storage container 6 for disposal. These steps proceed according to FIGS. 6 and 7.

One advantage of the method according to the invention is that it can comprise a procedural step, in which a lancet 5 that was used for a puncturing motion is returned to the lancet storage container 6. As a particular advantage, the invention allows the used lancets 5 to be returned to the cartridge without contamination of the lancet storage container 6 in that the used lancet 5 is transferred initially into the parking position before it is returned to the cartridge in the lancet storage container 6. Accordingly, it is proposed according to a preferred feature to insert the used lancet 5 in the parking position into a lancet tip protective element 13 prior to returning it to the lancet storage container 6, i.e. to initially transfer a used lancet 5 into the parking position even if it will not be provided for another puncturing process. The lancet storage container 6, possibly with used lancets 5 returned to the cartridge, can be replaced at a convenient time.

Therefore, the lancet storage container 6 serves only for the transport of the lancets 5 into the blood withdrawal system 1 and/or blood analysis device 2, for the provision of unused lancets therein, and for the disposal of the used lancets.

FIGS. 17 to 19 illustrate the alternative variant, in which the lancet drive 8 stays with the lancet 5 in the parking position after a blood taking process. FIG. 17 illustrates how the lancet 5 is transported into the parking position; the procedure corresponds to the procedure shown in FIG. 12. However, in contrast to FIGS. 13 and 14, subsequently, the lancet drive 8 is not driven or pivoted away from the lancet 5 in its parking position, but rather the grabber 9 stays at or in the immediate vicinity of the lancet 5 in its parking position. This is shown in FIG. 18. Compared to the final position shown in FIG. 14, this is advantageous in that it results in even shorter driving times for the parked lancet 5 for a subsequent blood taking process, since the driving paths shown in FIG. 15 are avoided. The steps illustrated in FIG. 19, which correspond to those of FIG. 16, are to be performed to perform another blood taking process. The disposal of a lancet 5 that is unfit for further use proceeds like in the first variant described above.

FIGS. 20 to 22 illustrate a modified embodiment of a blood analysis device 2 with a blood withdrawal system according to the invention. A test element cartridge 17 with a revolver-like removal facility 18 is situated on the top of the blood analysis device 2. The lancet storage container 6 is situated on its underside and can be replaced independent of the test element cartridge 17. In other embodiments, the lancets and test elements can be integrated into the same storage container.

The lancets 5 are driven from bottom to top through the lancet storage container 6 by a lancet drive that is not shown. Filled with a gel-like, soft plastic material, the lancet tip protective element 13 is arranged next to the lancet storage container 6. In other embodiments, the parking position that is defined by the position of the protective element 13 could occupy a position in the lancet storage container 6. A holding facility 19 serving as parking aid for lancets that are situated in the parking position is arranged underneath the protective element 13, for example in the form of a holding clamp. The procedure is illustrated in FIG. 21 in three steps. The lancet drive penetrates into a storage position of a lancet 5 in the lancet storage container 6 from below, whereby, for example, a sealing film/foil ensuring the sterility until this time is perforated. A lancet is then grabbed by a grabbing facility and driven in upward direction out of the lancet storage container 6, again, for example, through a sealing film/foil. After initiation of the puncturing, the lancet drive drives the lancet back to the lancet storage container 6, where, according to the prior art, it is separated from the lancet drive and disposed. For the next blood taking process, according to the prior art, the lancet storage container 6 would then rotate by one position and a new lancet would be used.

In contrast, according to FIG. 21 (middle), the used lancet 5 is pulled downwards through the lancet storage container 6 and then pivoted sideways and pushed upwards into the lancet tip protective element 13 by the lancet drive (FIG. 21, right side), where it is kept in store for a further use. For reusing a lancet from the parking position, the lancet is transported in the reverse sequence, i.e. first it is pulled down, then pivoted sideways and transported upwards through the lancet storage container 6.

FIG. 22 shows a lancet 5 in the lancet tip protective element 13 that is filled with a microbicidal elastic material 15. It is secured by a holding facility 19.

FIG. 23 illustrates a modified embodiment of the protective element 13. Possibly, a capillary (capillary vessel) is hit in the puncturing process (for collecting blood) and an unexpectedly large amount of blood exits. This amount of blood could collect on the lancet 5 in the form of a drop 20. This drop 20 would be wiped off on the surface of the elastic material 15 (of the parking gel) when the lancet 5 is punctured. From there, the still liquid blood might flow downwards or accumulate in the form of layers and henceforth contaminate the part of the lancet 5 that is not immersed into the elastic material. This could be remedied by providing the elastic material 15 with a cover 21 with an absorbent material, e.g. cellulose or similar substance. This can absorb the drop of blood 20, if any, and bind it intrinsically. The requirements on the material of the cover 21 include sufficient firmness, high density of the fabric (absorbent force and sufficient stability for several punctures), and non-pilling feature.

### LIST OF REFERENCE NUMBERS

1 Blood withdrawal system
2 Blood analysis device
3 Housing
4 Exit opening
5 Lancet
6 Lancet storage container
7 Rotation axis for 6
8 Lancet drive
9 Grabber
10 Rotation axis for 8
11 Linear axis for 8
12 Finger tip
13 Lancet tip protective element
14 Form body
15 Elastic material
16 Grabbing section
17 Test element cartridge
18 Removal facility
19 Holding facility
20 Drop
21 Cover

1. A blood withdrawal system for collecting blood for analytical or diagnostic purposes, comprising:
a housing with an exit opening for the lancet tip of the lancet needle of a lancet that can be is moved in the housing along a predetermined puncturing path,
a lancet guide which guides the lancet along the predetermined puncturing path,
a lancet drive which drives the lancet along the predetermined puncturing path in the puncturing direction until its tip, is in a puncturing position,
a lancet storage container arranged in the housing and in which a plurality of lancets stored at a removal position for removal from the lancet storage container,
a lancet tip protective element, into which the lancet tip can be inserted before or after a puncturing motion, whereby the protective element mechanically and hygienically protects a lancet tip that is inserted therein, and
the lancet tip protective element being arranged on the lancet tip in a parking position of the lancets, the parking position not coinciding with the removal position or the puncturing position.

2. The blood withdrawal system of claim 1, wherein the lancet tip protective element and the parking position are arranged proximate the exit opening.

3. The blood withdrawal system of claim 1, wherein the lancet tip protective element is stationary in the housing.

4. The blood withdrawal system of claim 1, wherein the lancet tip protective element is adapted to be driven onto the lancet tip.

5. The blood withdrawal system of claim 1, wherein the lancet can be driven by the lancet drive into the parking position, in which the lancet tip is situated in the lancet tip protective element.

6. The blood withdrawal system of claim 1, further comprising a holding facility for holding the lancet in the parking position.

7. The blood withdrawal system of claim 1, wherein the lancet tip protective element is arranged such that the lancet tip can be inserted into the lancet tip protective element by a motion that proceeds parallel to the puncturing motion.

8. The blood withdrawal system of claim 1, wherein the lancet tip protective element is arranged in the lancet storage container+.

9. The blood withdrawal system of claim 1, wherein the lancet tip protective element comprises an elastic material into which the lancet tip can be inserted.

10. The Bleed blood withdrawal system of claim 1, characterized in that the lancet tip protective element comprises a sterilizing, microbicidal, inactivating, disinfecting, bactericidal or fungicidal material for cleaning or protecting the lancet tip.

11. The blood withdrawal system of claim 9, wherein the elastic material comprises a sterilizing, microbicidal, inactivating, disinfecting, bactericidal or fungicidal material for cleaning or protecting the lancet tip.

12. The blood withdrawal system of claim 9, wherein the elastic material comprises a cover made of an absorbent material.

13. The blood withdrawal system of claim 1, wherein the lancet tip protective element is replaceable.

14. The blood withdrawal system of claim 1, wherein a lancet can be used repeatedly to collect multiple blood samples and can be driven into the lancet tip protective element between puncturing motions.

15. The blood withdrawal system of claim 1, further comprising operating elements the user can use to set whether a new lancet from the lancet storage container or a lancet from the parking position in the lancet tip protective element that was used previously for taking a blood sample is used for the subsequent blood taking process.

16. The blood withdrawal system of claim 1, further comprising a test element cartridge.

17. A blood analysis device, comprising a blood withdrawal system according to claims 1.

18. A method for drawing a blood sample with a blood withdrawal system comprising a housing with an exit opening for the lancet tip of the lancet needle of a lancet that is moved in the housing along a predetermined puncturing path and a lancet guide which guides the lancet along the predetermined puncturing path, a lancet drive which drives the lancet in the puncturing direction, and a lancet storage container that is arranged in the housing and in which a plurality of lancets is stored at a removal position for removal from the lancet storage container, the method comprising, inserting the lancet tip into a lancet tip protective element before or after a puncturing motion, whereby the protective element mechanically and hygienically protects a lancet tip that is inserted therein, and arranging the lancet tip protective element on the lancet tip in a parking position of the lancets, whereby the parking position does not coincide with the removal position and the puncturing position.

19. The method of claim 18, wherein the lancet tip protective element and the parking position are arranged proximate the exit opening.

20. The method of claim 18, wherein the lancet tip protective element is stationary in the housing.

21. The method of claim 18 further comprising driving the lancet tip protective element onto the lancet tip.

22. The method of claim 18 further comprising driving the lancet into the parking position, in which the lancet tip is situated in the lancet tip protective element.

23. The method of claim 18, further comprising holding the lancet in the parking position by a holding facility.

24. The method of claim 18, further comprising arranging the lancet tip protective element such that the lancet tip is inserted into the lancet tip protective element by a motion that proceeds parallel to the puncturing motion.

25. The method of claim 18, further comprising transporting a lancet that was used to perform a puncturing motion to the lancet storage container.

26. The method of claim 25, further comprising inserting the used lancet into a lancet tip protective element before transporting it back to the lancet storage container and placing it in the parking position.

27. The method of claim 18, further comprising replacing the lancet storage container.

28. The method of claim 18, wherein a lancet is used to take a multiple blood samples and is driven into the lancet tip protective element between the puncturing motions.
